(19) United States
(12) Patent Application Publication
(10) Pub. No.: US 2006/0121480 A1
(43) Pub. Date: Jun. 8, 2006

(54) Synergistic compositions for the prevention and treatment of acquired immunodeficiency syndrome

(75) Inventor: Sek Chung Fung, Houston, TX (US)

Correspondence Address: Cheryl Liljestrand;Tanox Inc, 10301 Stella Link Road, Houston, TX, 77025-5497, US

(73) Assignee: Tanox, Inc., Houston, TX (US)

(21) Appl. No.: 10/529,051

(22) PCT Filed: Sep. 26, 2003
(86) PCT No.: PCT/US03/30538

§ 371 Date: Mar. 24, 2005

Related U.S. Application Data
(60) Provisional application No. 60/414,062, filed on Sep. 27, 2002.

(51) Int. Cl. C12Q 1/68; C12P 21/06

(52) U.S. Cl. 435/6; 435/69.1

(57) ABSTRACT

A method for preventing infection of helper T and other target cells by human immunodeficiency virus type 1 (“HIV-1”) and for preventing or treating acquired immunodeficiency syndrome (“AIDS”) by exposing target cells to a synergistic combination of at least one attachment inhibitor and at least one fusion inhibitor. The attachment inhibitors are compounds that bind to the CD4 receptor on target cells or that bind to gp120 on HIV-1, e.g., antibodies, and the fusion inhibitors compounds that interact with gp41 to inhibit or prevent its interaction with target cells, e.g., pentafuside.

### BACKGROUND OF THE INVENTION

1. Field of the Invention

This invention relates generally to methods and compositions for the prevention and treatment of acquired immunodeficiency syndrome.

2. Description of the Prior Art

Acquired immunodeficiency syndrome (“AIDS”) is a disease characterized by a weakened immune system that has difficulty combating opportunistic infections. These opportunistic infections that cause severe disease are life threatening for individuals with AIDS but are usually controlled by a non-infected individual with a healthy immune system. Unfortunately, the immune system of an individual suffering from AIDS is often so weak that medical intervention is required to control the disease or prevent death.

AIDS is principally caused by a retrovirus known as the human immunodeficiency virus type 1 (“HIV-1”). HIV-1 weakens the immune system by invading the body and then infecting and depleting helper T cells. Helper T cells are essential to a healthy immune system because they control the production of antibodies by B cells, maturation of cytotoxic T lymphocytes (killer T cells), maturation and activity of macrophages and natural killer cells, and numerous other regulator and effector functions of the immune system.

Infection and depletion of helper T cells occurs through a multistep process that requires viral attachment to the CD4 receptor of helper T cells, viral attachment to coreceptor CXCR4 or CCR5, viral fusion with the cell, viral uncoating, reverse transcription of viral RNA to form DNA, synthesis of a second strand of DNA, migration of the DNA to the helper T cell nucleus, integration of viral DNA into the helper T cell genome, transcription of the DNA to produce RNA, translation of viral RNA to produce a viral polyprotein, viral protease cleavage of the polyprotein to produce viral proteins, and assembly and budding of the viral proteins to form new virus and destroy the host cell. Different drugs and treatment methods have been designed to interfere with one or more of these steps. Typical methods used to prevent or treat the disease include the use of compounds that inhibit the attachment of the virus to the helper T cell or other target cells such as macrophages (attachment inhibitors), inhibit the fusion of the virus with the target cell (fusion inhibitors), prevent the integration of the viral DNA into the helper T cell DNA (integrase inhibitors), prevent the synthesis of DNA from viral RNA (reverse transcriptase inhibitors), and prevent the cleavage of the viral polyprotein into viral proteins by proteases (protease inhibitors).

Some of these compounds have been shown to behave synergistically, e.g., Beale, K. K. and Robinson, W. E., Jr. Combinations of reverse transcriptase, protease, and integrase inhibitors can be synergistic in vitro against drug-sensitive and RT inhibitor-resistant molecular clones of HIV-1. Antiviral Res. 46:223-232 (2000) and Essey, R. J., McDougall, B. R., and Robinson, W. E., Jr. Mismatched double-stranded RNA (poly I-poly C12U) is synergistic with different classes of anti-HIV drugs and against drug-sensitive and drug-resistant HIV-1 in vitro. Antiviral Res., 51:189-202 (2001).

However, AIDS has proven difficult to effectively prevent or treat. The virus has the ability to mutate and develop resistance to drugs used to treat the disease, including additive and synergistic drug combinations. Because of this difficulty, there has been a continuous need to find new drugs and methods for treating the disease.

Recent interest has focused on the use of attachment inhibitors and fusion inhibitors to prevent HIV-1 infection rather that the use of drugs such as reverse transcriptase or protease inhibitors that function after infection, i.e., Program and Abstracts of the XW International AIDS Conference. Jul. 7-12, 2002. Barcelona, Spain. “Greenberg. Fusion Inhibitors. Abstract MoOrAI39” and “W. Olson and others. The Attachment and CCR5 Inhibitors PRO 542 and PRO 140. Abstract MoOrA140.” Indeed, attachment and fusion inhibitors have been found to be effective in preventing helper T cell infection by HIV-1.

HIV-1 entry into helper T cells or macrophages occurs in distinct steps, i.e., attachment and fusion. Attachment and fusion require the interaction of several viral and cellular proteins in distinct phases: (1) attachment of viral envelope proteins to the primary receptor CD4, (2) conformational change in the viral proteins that result in the binding to a coreceptor, and (3) exposure of viral proteins that result in the fusion of the viral and target cell membranes. Attachment and fusion are principally mediated by the viral proteins gp120 and gp41. Gp120 and gp41 form a complex that is present as a trimer on the virion surface. Gp120 is the viral protein that attaches to the primary receptor CD4 on the surface of target cells. Gp120 attachment brings the virus and cell into contact although not in sufficient contact to initiate fusion. The extracellular region of CD4 consists of 4 domains (D1, D2, D3, and D4). The HIV-1 gp120 binding site on CD4 comprises amino acids 40 to 60 (strand C′, C″, and D) in the CD4 domain 1 (D1), a stretch analogous to the CDR2 of an immunoglobulin (Ig) V domain. After attachment of gp120 to CD4, gp120 undergoes conformational change and thus binds to a chemokine coreceptor (CCR5 or CXCR4). CCR5 is the chemokine receptor used by macrophage-tropic and certain T-cell-tropic primary HIV-1 isolates while most T-cell tropic primary HIV-1 isolates and T-cell line-adapted HIV-1 strains use CXCR4. Some T-cell tropic isolates are dual tropic that can use either CCR5 or CXCR4 as a coreceptor. After the interaction between HIV-1 gp120 and the co-receptors, HIV-1 gp41 is exposed. Gp41 then undergoes a harpoon-like conformational change that forms an attachment to the target cell membrane and then uses a spring-like mechanism to form a triple helical, u-shaped protein structure known as the “trimer of hairpins. Forming the hairpin structure draws the virus to the cell and initiates membrane fusion. This fusion results in the viral particle entering into the target cell and subsequently infect the cell.

Attempts to prevent HIV-1 infection by inhibiting attachment have proved successful. U.S. Pat. No. 6,309,880 issued to Chang, et al. on Oct. 30, 2001 (assigned to Tanox, Inc. (Houston, Tex.)) entitled “Antibodies specific for CD4-binding domain of HIV-1” discloses a particular epitope located within the CD4-binding region of HIV-1 gp120 of and antibodies specific for the epitope that can inhibit HIV-1 infection of human cells by diverse viral strains and isolates. U.S. Pat. No. 5,871,732 issued to Burkly, et al. on Feb. 16, 1999 (assigned to Biogen, Inc. (Cambridge, Mass.)) entitled “Anti-CD4 antibody homologs useful in prophylaxis and treatment of AIDS, ARC and HIV infection” discloses anti-CD4 antibody homologs useful for preventing or treating diseases in mammals, including AIDS. U.S. Pat. No. 5,817,767 issued to Allaway, et al. on Oct. 6, 1998 (assigned to Progenics Pharmaceuticals, Inc. (Tarrytown, N.Y.)) entitled “Synergistic composition of CD4-based protein and anti-HWV-1 antibody, and methods of using same” discloses compositions containing CD4-based immunoconjugates and antibodies specific for the envelope glycoprotein of HIV-1. The compositions of the invention act synergistically to neutralize HIV-1. U.S. Pat. No. 5,922,325 issued to Tilley, et al. on Jul. 13, 1999 (assigned to Public Health Research Institute of the City of New York, Inc. (New York, N.Y.)) entitled “Synergistic neutralization of HIV-1 by human monoclonal antibodies and other antibodies directed against the V3 loop and the CD-4 binding site of GP-120, and the use for immunotherapy of HIV-1 infection” discloses a synergistic combination of antibodies specific for H-1 envelope glycoprotein gp120. One of the antibodies specific for the V3 loop and the other is specific for the CD-4 binding site of gp120. U.S. Pat. No. 6,241,986 issued to Zolla-Pazner, et al. on Jun. 5, 2001 (assigned to New York University (New York, N.Y.)) entitled “Human monoclonal antibodies to the CD-4 binding domain of HIV, uses thereof and synergistic neutralization of HIV” discloses monoclonal antibodies specific for the CD4-binding domain of HIV gp120 that are useful in the neutralization of HIV-1 and in the prevention of HIV infection and the treatment of a subject infected with HIV. U.S. Pat. No. 6,136,310 issued to Hanna, et al. on Oct. 24, 2000 (assigned to IDEC Pharmaceuticals Corporation (San Diego, Calif.)) entitled “Recombinant anti-CD4 antibodies for human therapy” discloses chimeric antibodies specific to human CD4 antigen.

Attempts to prevent HIV infection by inhibiting fusion have also proved successful. U.S. Pat. Nos. 6,015,881 and 6,281,331 issued to Kang, et al. on Jan. 18, 2000 and Aug. 28, 2001, respectively, (assigned to Trimeris, Inc.) entitled “Methods and compositions for peptide synthesis” discloses peptides T-20 and related peptides useful for the treatment of HIV-1 infections. T-20, also known as pentafuside, is a 36 amino acid peptide that prevents fusion between HIV-1 and target cells in vitro and in vivo. T20 and its analogues are derived from the C-terminus peptide segment of HIV-1 gp41. This peptide segment is involved in the interaction with its N-terminus peptide counterpart of HIV-1 gp41. The binding of these C-and N-terminus peptides results in formation of trimer-of-hairpins which are essential for the fusion between the membrane of target cells and HIV-1 viral coat (Sodroski, Cell 1999; 99: 243-6). Pentafuside interferes with the formation of the trimer-of-hairpins. Pentafuside is being developed as Fuzeon™ brand enfuvirtide (T-20) by Trimeris and Hoffmann-La Roche. A 39 amino acid peptide known as T-1249 is a fusion inhibitor with similar function.

A protein known as 5-Helix inhibits the formation of the trimer-of-hairpins by using the N-terminus peptide segment of HIV-1 gp41 to block the C-terminus peptide segment of HIV-1 gp41. The protein 5-Helix is made of alternatively linked N- and C-terminus peptide segments (N—C—N—C—N) of gp41. This structure is constituted in such a way that it will not cause aggregation of the protein molecules (Root et al., Sci. 2001; 291: 884-8).

Treatment for AIDS using attachment and fusion inhibitors as well as other antiviral drugs has been effective. Current clinical treatments for HIV-1 infections include triple drug combinations called Highly Active Antiretroviral Therapy (“HAART”). HAART typically involves various combinations of nucleoside reverse transcriptase inhibitors, non-nucleoside reverse transcriptase inhibitors, and HIV-1 protease inhibitors. In compliant patients, HAART is effective in reducing mortality and progression of HIV-1 infection to AIDS. However, these multidrug therapies do not eliminate HIV-1 and long-term treatment often results in multidrug resistance. Also, many of these drugs are highly toxic and/or require complicated dosing schedules that reduce compliance and limit efficacy. There is, therefore, a continuing need for the development of additional drugs for the prevention and treatment of HIV-1 infection and AIDS. Ideally, these drugs would target early stages in the HIV-1 infective life cycle, i.e., inhibit or prevent attachment and fusion.

### SUMMARY OF THE INVENTION

It is, therefore, an object of the invention to provide methods and compositions for preventing or treating HIV-1 infection of target cells such as helper T cells and macrophages.

It is another object of the invention to provide methods and compositions for preventing or treating AIDS.

These and other objects are achieved by exposing target cells to a synergistic combination of at least one attachment inhibitor such as an antibody that binds to CD4 and at least one fusion inhibitor such as pentafuside. The combination is useful because its synergistic actions permits the use of less drug or increases the efficacy of the drugs when used together in the same amount as when used alone.

Other and further objects, features, and advantages of the present invention will be readily apparent to those skilled in the art.

### DETAILED DESCRIPTION OF THE INVENTION

### Definitions

The term “patient” means a primate susceptible to infection by HIV-1 and the development of AIDS. Preferably, the primate treated according to the present invention is a human.

The term “in conjunction” means that attachment inhibitors and fusion inhibitors of the present invention are administered to a patient (1) separately at the same or different frequency using the same or different administration routes or (2) together in a pharmaceutically acceptable composition.

The term “parenterally” means administration by intravenous, subcutaneous, intramuscular, or intraperitoneal injection or administration by subcutaneous implant.

The term “synergism” means a cooperative effect between individual compounds such that the total effect is greater than the sum of the effects of the compounds taken independently.

The term “functionally equivalent peptides” means a fragment of a polypeptide that has the same biological activity as the polypeptide.

The term “target cell(s)” means any cell expressing CD4 and/or chemokine co-receptors CCR5 or CXCR4 on the cell membrane that HIV-1 can attach and infect, e.g., helper T cells and macrophages, or any cell that can be infected by fusion between non-infected cells and HIV-1 infected cells expressing HIV-1 gp120 and gp41 on the cell membrane.

This invention is not limited to the particular methodology, protocols, and reagents described herein because they may vary. Further, the terminology used herein is for the purpose of describing particular embodiments only and is not intended to limit the scope of the present invention. As used herein and in the appended claims, the singular forms “a,” “an,” and “the” include plural reference unless the context clearly dictates otherwise, e.g., reference to “a host cell” includes a plurality of such host cells.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, devices, and materials are described herein.

All patents and publications mentioned herein are incorporated herein by reference to the extent allowed by law for the purpose of describing and disclosing the antibodies, polypeptides, peptides and methodologies reported therein that might be used with the present invention. However, nothing herein is to be construed as an admission that the invention is not entitled to antedate such disclosure by virtue of prior invention.

### The Invention

In one aspect, the present invention provides a method for preventing infection of target cells by human immunodeficiency virus type 1 (“HIV-1”). The method comprises exposing target cells to an infection preventing amount of a synergistic combination of at least one attachment inhibitor and at least one fusion inhibitor. The method is useful for preventing or treating AIDS in patients who are at high risk for acquiring the disease or who have acquired the disease after exposure to HIV-1.

The attachment inhibitors of the present invention are polypeptides or other compounds that bind to the CD4 receptor on target cells or that bind to gp120 on HIV-1 and inhibit or prevent HIV-1 from attaching to the target cells or permit HIV-1 to attach to the target cells but inhibit or prevent cellular fusion between HIV-1 and the target cells. Generally, the attachment inhibitors are antibodies, antibody fragments, CD4 antagonists comprising a fragment of a CD4 ligand such as gp120, or gp120 antagonists comprising a fragment of CD4 such as a fusion protein of CD4 with human IgG2. Preferably, the attachment inhibitors are polyclonal or monoclonal antibodies that bind to gp120 and prevent attachment of gp120 to CD4 or permit attachment of gp120 to CD4 but inhibit or prevent fusion of the virus and the target cell and polyclonal or monoclonal antibodies that bind to CD4 and prevent attachment of gp120 to CD4 or permit attachment of gp120 to CD4 but inhibit or prevent fusion of the virus and the target cell. Most preferably, the attachment inhibitors are monoclonal antibodies that bind to CD4 and permit attachment of gp120 but inhibit or prevent fusion of HIV-1 and the target cell, including, but not limited to, the antibodies disclosed in U.S. Pat. No. 5,871,732.

The fusion inhibitors of the present invention are polypeptides or other compounds that interact with gp41 to inhibit or prevent its harpoon-like binding to target cells or inhibit or prevent its recoil-like action that brings HIV-1 into close contact with target cells. Preferably, the fusion inhibitors are polypeptides that interact with gp41 to prevent its harpoon-like action that binds gp41 to target cells or its recoil-like action that brings HIV-1 into close contact with target cells. Most preferably, the fusion inhibitors are anti-gp41 antibodies or smaller polypeptides having from 30 to 50 amino acids and the ability to interact with gp41 to prevent its harpoon-like or recoil-like action. In one embodiment, the fusion inhibitor is a polypeptide selected from the group consisting of the 39 amino acid polypeptide known as T-1249, the 36 amino acid polypeptide known as T-649, the protein 5-Helix, or their functionally equivalent peptides. Most preferably, the fusion inhibitor is pentafuside (T-20) and its functionally equivalent peptides.

In one embodiment, the method comprises exposing target cells to a synergistic combination of an anti-CD4 antibody that binds to CD4 and inhibits or prevents the attachment of gp120 to CD4 and pentafuside or its functionally equivalent peptides. Several of these antibodies are known in the art or such antibodies can be made by techniques known in the art, e.g., the antibodies disclosed in U.S. Pat. Nos. 5,961,576 and 5,912,176.

In another embodiment, the method comprises exposing target cells to a synergistic combination of an anti-CD4 antibody that permits the binding of gp120 to the CD4 receptor but inhibits the infection of target cells by HIV-1 and pentafuside or its functionally equivalent peptides. Several of these antibodies are known in the art or such antibodies can be made by techniques known in the art, e.g., the antibodies disclosed in U.S. Pat. No. 5,871,732.

In another embodiment, the method comprises exposing target cells to a synergistic combination of an anti-HIV-1 gp120 antibody that binds to gp120 and inhibits or prevents the attachment of gp120 to CD4 and pentafuside or its functionally equivalent peptides. In this embodiment, the attachment inhibitors are preferably monoclonal antibodies that bind to the CD4 binding site on gp120. Several of these antibodies are known in the art or such antibodies can be made by techniques known in the art, e.g., the antibodies disclosed in U.S. Pat. No. 6,309,880 and U.S. Pat. No. 6,241,986.

In another embodiment, the method comprises exposing target cells to a synergistic combination of an anti-HIV-1 gp120 antibody that permits the binding of gp120 to the CD4 receptor but inhibits the infection of target cells by HIV-1 and pentafuside or its functionally equivalent peptides. The anti-HIV-1 gp120 antibody may bind to any epitope on gp120, including the binding site for chemokine co-receptor CCR5 or CXCR4. Several of these antibodies are known in the art or such antibodies can be made by techniques known in the art, e.g., the antibodies disclosed in U.S. Pat. No. 5,922,325.

In another embodiment, the method comprises exposing target cells to a synergistic combination of an anti-HIV-1 co-receptor antibody that binds to the chemokine co-receptor CCR5 or CXCR4 and inhibits or prevents the attachment of the co-receptor to gp120 and pentafuside or its functionally equivalent peptides. Several of these antibodies are known in the art or such antibodies can be made by techniques known in the art.

The polypeptides of the present invention can be designed and produced using protein modeling methods known in the art. Many computational algorithms for designing and/or modeling protein conformations are described in the art, e.g., WO 98/47089.

The attachment inhibitors and fusion inhibitors can be administered in combination with other drugs such as integrase inhibitors, nucleoside reverse transcriptase inhibitors, non-nucleoside reverse transcriptase inhibitors, and HIV protease inhibitors, including in HAART-like treatments.

In another embodiment, the method further comprises exposing target cells to the attachment inhibitors and fusion inhibitors of the present invention in combination with at least one other drug such as integrase inhibitors, nucleoside reverse transcriptase inhibitors, non-nucleoside reverse transcriptase inhibitors, and HIV protease inhibitors. Preferably the drug is at least one integrase inhibitor, and/or at least one transcriptase inhibitor, and/or at least one protease inhibitor. Such methods are useful in HAART regiments. Most preferably, the method comprises exposing target cells to at least one anti-CD4 or anti-gp120 antibody and pentafuside and one or more integrase, transcriptase, or protease inhibitors. The anti-gp120 antibody is preferably an antibody disclosed in U.S. Pat. No. 6,309,880. The anti-CD4 antibody is preferably an antibody disclosed in U.S. Pat. No. 5,871,732.

In another aspect, the present invention provides a method for preventing or treating acquired immunodeficiency syndrome (“AIDS”). The method comprises administering a disease preventing or treating amount of a synergistic combination of at least one attachment inhibitor and at least one fusion inhibitor to a patient at risk for contracting or suffering from AIDS.

In a further aspect, the present invention provides a composition useful for preventing infection of target cells by HIV-1 and for preventing or treating AIDS comprising at least one attachment inhibitor and at least one fusion inhibitor. The composition comprises the inhibitors alone or in combination with pharmaceutically acceptable carriers such as various vehicles, adjuvants, additives, and diluents. The composition is useful for preventing or treating AIDS.

The attachment inhibitors and fusion inhibitors of the present invention can be administered or coadministered to a patient by any suitable method known in the art, particularly for administering peptides or polypeptides. Such methods include, but are not limited to, injections, implants, and the like. Injections are preferred because they permit precise control of the timing and dosage levels used for administration. The inhibitors can be administered parenterally, intraperitoneally, intravenously, intraarterially, transdermally, sublingually, intramuscularly, subcutaneously, intraarticularly, or intrathecally. The inhibitors are preferably administered parenterally.

Preferably, the attachment inhibitors and fusion inhibitors are administered at about the same time but, if administration is difficult, the inhibitors are effective if administered in conjunction. For example, the attachment inhibitor can be administered by intravenous injection and the fusion inhibitor can be administered by intramuscular injection into the patient within a reasonable time, generally within 8 hours, preferably within 2 hours, and most preferably within 0.1-0.5 hours. Many such administration patterns will be apparent to those skilled in the art.

The present invention encompasses the use of a single attachment inhibitor and a single fusion inhibitor, the use of a single attachment inhibitor and two or more fusion inhibitors, the use of a two or more attachment inhibitor and a single fusion inhibitor, and the use of two or more attachment inhibitors and two or more fusion inhibitors, all in combination with multiple other drugs in combination therapy for the treatment of AIDS.

The attachment inhibitors and fusion inhibitors can be administered in a single dose or can be administered in multiple doses over a defined period. For example, the attachment inhibitors can be administered by intravenous injection as a single, dose and the fusion inhibitor can be administered by daily injection over a period of several days. Many such administration patterns will be apparent to those skilled in the art.

The amount or dosage of attachment inhibitors and fusion inhibitors administered may vary depending upon the age, size, and health of the patient, the administration pattern, the severity of the disease, and whether the dose is to act therapeutically or prophylactically. Generally, attachment inhibitors are administered to the patient in dosages of from about 1 to 50 milligrams per kilogram of body weight (mg/kg), preferably from about 5 to 30 mg/kg, and the fusion inhibitors are typically administered to the patient in dosages of from about 0.1 to 10 milligrams per kilogram of body weight (mg/kg), preferably from about 0.5 to 5 mg/kg. The attachment inhibitors are typically administered on a weekly schedule but may be administered on a daily schedule. The fusion inhibitors are typically administered daily but may be administered multiple times per day. For repeated administrations over several days, weeks, or longer, depending on the condition, the treatment is repeated until a desired suppression of HIV-1 viral load and/or disease symptoms occurs or the desired improvement in the patient's condition is achieved. The dosage may be readministered at intervals ranging from once a week to once every six months. The determination of the optimum dosage and of optimum route and frequency of administration is well within the knowledge of those skilled in the art. Similarly, dosages for other drugs within the scope of the present invention can be determined without excessive experimentation.

The compositions of the present invention include pharmaceutically acceptable carriers that are inherently nontoxic and nontherapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol. Such pharmaceutical compositions may be prepared and formulated in dosage forms by methods known in the art.

In another aspect, since the attachment inhibitors and fusion inhibitors can be administered separately or with other drugs, the present invention provides an article of manufacture in the form of a kit comprising in separate containers in a single package a combination of two or more of (1) an attachment inhibitor, (2) a fusion inhibitor, and, optionally, (3) another drug useful for inhibiting or preventing HIV-1 infection of target cells or for the prevention or treatment of AIDS. The kit contains attachment inhibitors in amounts sufficient to supply from about from about 1 to 50 milligrams per kilogram of body weight (mg/kg), preferably from about 5 to 30 mg/kg, and the fusion inhibitors are typically administered to the patient in dosages of from about 0.1 to 10 milligrams per kilogram of body weight (mg/kg), preferably from about 0.5 to 5 mg/kg when administered to a patient. Amounts of other drugs to include in the kit are determined by reference to approved or recommended dosages for the particular drug. Typically, the kit contains one attachment inhibitor and one fusion inhibitor. Preferably, the kit contains an anti-CD4 antibody, anti-HIV-1 gp120 antibody, or anti-HIV-1 gp41 antibody, and pentafuside. Most preferably, the kit contains an anti-CD4 antibody and pentafuside. Preferably, the optional drugs are integrase, transcriptase, or protease inhibitors.

In another aspect, the present invention provides a means for communicating information about or instructions for synergistically using attachment inhibitors and fusion inhibitors to prevent infection of target cells by HIV-1 and to prevent or treat AIDS. The communicating means comprises a document or visual display that contains the information or instructions. Preferably, the communication is a web site displayed on a visual monitor, brochure, or package insert containing such information or instructions.

Useful information includes the fact that the inhibitors are synergistic, details about the side effects, if any, caused by using the inhibitors in combination and in combination with other drugs, and contact information for patients to use if they have a question about the inhibitors or their use. Useful instructions include inhibitor dosages, administration amounts and frequency, and administration routes. The communication means is useful for instructing a patient on the benefits of using the synergistic inhibitors of the present invention and communication the approved methods for administering the inhibitors to a patient.

### EXAMPLES

This invention can be further illustrated by the following examples of preferred embodiments thereof, although it will be understood that these examples are included merely for purposes of illustration and are not intended to limit the scope of the invention unless otherwise specifically indicated.

### Materials and Methods

Viruses: 6 viruses were titrated. The TCID₅₀/ml of each virus isolate was as the follows:

| | |
|---|---|
| HIV-1 302076 (pediatric): 39810; | HIV-1 302077 (pediatric): 39810; |
| HIV-1 302143 (pediatric): 158489; | HIV-1 2054 (adult): 19952; |
| HIV-1 301714 (adult): 10000; | NIH. HTLV-IIIB (lab): 5011 |

Virus stocks were diluted to 2,000 TCID₅₀/ml.

512.2 μl of HIV-1 302076 was added into 9487.8 μl R-3 medium. 512.2 μl of HIV-1 302077 was added into 9487.8 μl R-3 medium. 126.3 μl of HIV-1 302143 was added into 9873.7 μl R-3 medium. 1002.4 μl of HIV-1 301714 was added into 8997.6 R-3 medium. 2000 μl of HIV-1m 2054 was added into 8000.0 μl R-3 medium. 4000 μl of HTLV-IIIB was added into 6000 R-3 μl medium. Viruses were diluted to 100 TCID50 virus stock: 2.5 ml of 2000 TCID₅₀/ml was added to 2.5 ml of R-3 medium, this is 100 TCID50 (in 100 μl) stock. PBMC: Peripheral blood mononuclear cells (PBMC) were separated from HI-1-uninfected donor by Ficoll-hypaque density gradient centrifugation. PBMC were grown in RPMI 1640 supplemented with 20% fetal calf serum, 5% IL-2 and 5 μg/ml PHA (R-3 medium).

5A8 (humanized monoclonal antibody to CD4): The preparation was in sterile PBS, PH 7.0, at 11.55 mg/ml. The antibody was stored under sterile condition at 4-8° C. HPLC analysis showed that the antibody was monometric and had a purity of over 99%. 5A8 to stock solution was diluted (500 μg/ml).

60 μl of 5A8 (11.55 mg/ml) was added to 1326 μl PBS, this was the 5A8 stock solution (500 μg/ml). 5A8 stock solution was diluted to concentrations used in the experiments: 192 μl of 5A8 stock solution was added to 1008 μl of PBS, this is the 80 μg/ml solution (1; 6.25 dilution). 200 μl of 5A8 80 μg/ml was taken and 800 μl PBS (1:31.25 dilution) was added. The above step was repeated for 1:156.25, 781.25, 3906.25 and 19531.25 dilutions. Finally, the concentrations used in the experiment were 2, 0.4, 0.08, 0.016, 0.0032, 0.00064 μg/ml.

T-20: The preparation had a purity of over 96.55% T-20 was stored at −20° C. and protected from light. T-20 to stock solution was diluted: 5 mg of T-20 was dissolved in 5 ml of PBS, this was the T-20 stock dilution (1 mg/ml).

T-20 was diluted to concentrations used in the experiments: 48 μl of T-20 stock solution was added to 1152 μl of PBS, this was the 40 μg/ml Solution (1:25 dilution). 200 μl of T-20 40 μg/ml was taken and 800 μl PBS (1:125 dilution) was added. The above step was repeated for 1:625, 1:3125, 1:15625, 1:78125 and 390625 dilution. Finally, the concentrations used in the experiment were 1. 0.2.0.04, 0.008, 0.0016, 0.00032 μg/ml.

### Example 1

### Drug Exposure at Infection and for 12 Hours Thereafter

Virus, cells, and agents were incubated at 37° C. overnight as follows: Total of 18 tubes for 6 5A8 dilutions, 3 tubes for virus control; Total of 18 tubes for 6 T-20 dilutions, 3 tubes for virus control; and Total of 18 tubes for 6 5A8/T-20 dilutions, 3 tubes for virus control.

50 μl 5A8 concentrations and 50 μl T-20 concentrations were added into the first 18 tubes starting with the highest concentration. The final three tubes are added to 100 μl PBS. 50 μl T-20 concentrations and 50 μl PBS was added into the second 18 tubes starting with the highest concentration. The final 3 tubes are added with 100 μPBS. 50 μl 5A8 and 50 μl PBS was added into the third 18 tubes starting with the highest concentration. The final 3 tubes are added to 100 μl PBS. 100 μl of 100 TCID₅₀ virus stock were aliquoted into all 63 tubes. 2×10⁶ PBMC was added in 1.8 ml of R-3 medium (total 2.0 ml per tube). The tubes were incubated at 37° C. overnight.

The cells were washed 3 times in PBS. The cells were resuspended in 2 ml R-3 medium in 24 well-plate without the addition of new 5A8 or T-20. HIV-1 P24 antigen measurement was performed in the supernatant of each coculture well on days 4 and 7.0.5 ml cells (10⁶/ml) in R-3 medium were added on day 4. The IC₅₀ and Combination Index were calculated using “Chou Dose Effect.” The results are shown in Table 1.

TABLE 1

| | | | | | | |
|---|---|---|---|---|---|---|
| | | | 5A8/ | *CI | T-20/ | *CI |
| Viral Strain | 5A8 | T-20 | T-20 | (IC₅₀) | 5A8 | (IC₅₀) |

IC₅₀ (μg/ml) and *CI (day 4)

| | | | | | | |
|---|---|---|---|---|---|---|
| HIV-1 302076 | 0.15 | 0.043 | 0.032 | 0.33 | 0.016 | 0.12 |
| HIV-1 302077 | 0.17 | 0.016 | 0.017 | 0.41 | 0.0081 | 0.20 |
| HIV-1 302143 | 0.97 | 0.14 | 0.13 | 0.21 | 0.065 | 0.20 |
| HIV-1 2054 | 0.070 | 0.039 | 0.015 | 0.36 | 0.0077 | 0.14 |
| HIV-1 301714 | 0.70 | 0.20 | 0.11 | 0.28 | 0.053 | 0.14 |
| HTLV-IIIB | 0.44 | 0.011 | 0.025 | 0.73 | 0.012 | 0.38 |

IC50 (μg/ml) and *CI (day 7)

| | | | | | | |
|---|---|---|---|---|---|---|
| HIV-1 302076 | 0.65 | 0.29 | 0.15 | 0.33 | 0.078 | 0.17 |
| HIV-1 302077 | 0.50 | 0.071 | 0.14 | 0.84 | 0.071 | 0.42 |
| HIV-1 302143 | >2.0 | 0.66 | 1.04 | 0.87 | 0.52 | 0.44 |
| HIV-1 2054 | 0.13 | 0.11 | 0.088 | 0.72 | 0.044 | 0.36 |
| HIV-1 301714 | 1.82 | 0.050 | 0.043 | 0.31 | 0.021 | 0.15 |
| HTLV-IIIB | 1.30 | 0.015 | 0.017 | 0.39 | 0.0087 | 0.20 |

### Example 2

### Continuous Drug Exposure

Example 1 was repeated except that concentrations of T-20 and 5A8 were changed as follows: T-20: 0.1, 0.02, 0.004, 0.0008, 0.00016, 0.000032 μg/ml; 5A8: 1.0, 0.2, 0.04, 0.008, 0.0016, 0.00032 μg/ml. The results are shown in Table 2.

TABLE 2

| | | | | | | |
|---|---|---|---|---|---|---|
| | | | 5A8/ | | T-20/ | |
| Viral Strain | 5A8 | T-20 | T-20 | *CI | 5A8 | *CI |

IC₅₀ (μg/ml) and *CI (day 4)

| | | | | | | |
|---|---|---|---|---|---|---|
| HIV-1 302076 | 0.10 | 0.0070 | 0.014 | 0.50 | 0.00053 | 0.11 |
| HIV-1 302077 | 0.14 | 0.0069 | 0.015 | 0.31 | 0.0016 | 0.031 |
| HIV-1 302143 | 0.038 | 0.044 | 0.0045 | 0.12 | 0.00045 | 0.012 |
| HIV-1 2054 | 0.041 | 0.019 | 0.025 | 0.67 | 0.0023 | 0.062 |
| HIV-1 302174 | 0.14 | 0.049 | 0.070 | 0.58 | 0.0070 | 0.058 |
| HTLV-IIIB | 0.19 | 0.017 | 0.0068 | 0.69 | 0.0068 | 0.069 |

IC50 (μg/ml) and *CI (day 7)

| | | | | | | |
|---|---|---|---|---|---|---|
| HIV-1 302076 | 0.028 | 0.0036 | 0.013 | 0.75 | 0.0013 | 0.075 |
| HIV-1 302077 | 0.067 | 0.0065 | 0.015 | 0.41 | 0.0015 | 0.041 |
| HIV-1 302143 | 0.022 | 0.021 | 0.0060 | 0.27 | 0.0060 | 0.27 |
| HIV-1 2054 | 0.017 | 0.16 | 0.0092 | 0.49 | 0.0016 | 0.049 |
| HIV-1 301714 | 0.30 | 0.040 | 0.097 | 0.51 | 0.0097 | 0.052 |
| HTLV-IIIB | 0.16 | 0.013 | 0.042 | 0.31 | 0.0043 | 0.031 |

*CI: Combination Index: CI < 0.9 indicates synergism; 0.9 <CI < 1.1 indicates additive interactions; CI > 1.1 indicates antagonism.

Referring to Tables 1 and 2, the data shows that there is synergistic activity between 5A8 and T-20 against HIV-1 replication in vitro under all conditions studied. All calculated CI values are less than 0.9 and IC₅₀'s are decreased 1-10 fold in the presence of both agents. In general, 5A8 adds more to the activity of T-20 than vice versa. When concentrations of 5A8 and T-20 are maintained for 7 days, IC₅₀ of 5A8 and T-20 decrease several fold compared to conditions in which drugs are removed 12 hours after the addition of infectious virus to target cells.

In the specification, there have been disclosed typical preferred embodiments of the invention and, although specific terms are employed, they are used in a generic and descriptive sense only and not for purposes of limitation, the scope of the invention being set forth in the following claims. Obviously many modifications and variations of the present invention are possible in light of the above teachings. It is therefore to be understood that within the scope of the appended claims the invention may be practiced otherwise than as specifically described.

What is claimed is:

1. A method for preventing infection of target cells by human immunodeficiency virus type 1 (“HIV-1”) comprising exposing target cells to an infection preventing amount of a synergistic combination of at least one attachment inhibitor and at least one fusion inhibitor.

2. The method of claim 1 wherein the attachment inhibitor is a polypeptide selected from the group consisting of polypeptides that bind to the CD4 receptor on target cells and inhibit or prevent HIV-1 from attaching to the target cell, polypeptides that bind to the CD4 receptor on target cells and permit HIV-1 to attach to the target cell but inhibit or prevent cellular fusion between HIV-1 and the target cell, polypeptides that bind to gp120 on HIV-1 and inhibit or prevent HIV-1 from attaching to the target cells, and polypeptides that bind to gp120 on HIV-1 and permit HIV-1 to attach to the target cell but inhibit or prevent cellular fusion between HIV-1 and the target cell and the fusion inhibitor is selected from the group consisting of polypeptides that interact with gp41 to inhibit or prevent its harpoon-like attachment to target cells and polypeptides that interact with gp41 to inhibit or prevent its recoil-like action that brings HIV-1 into close contact with target cells.

3. The method of claim 2 wherein the attachment inhibitor is selected from the group consisting of antibodies, antibody fragments, CD4 antagonists comprising a fragment of a CD4 ligand, and gp120 antagonists comprising a fragment of CD4 and the fusion inhibitor is selected from the group consisting of anti-gp41 antibodies and polypeptides having from 30 to 50 amino acids and the ability to interact with gp41 to prevent its harpoon-like or recoil-like action.

4. The method of claim 3 wherein the attachment inhibitor is selected from the group consisting of anti-CD4 antibodies and anti-gp120 antibodies and the fusion inhibitor is selected from the group consisting of T-1249, T-649, 5-Helix, pentafuside, and their functionally equivalent peptides.

5. The method of claim 2 wherein the attachment inhibitor is an anti-CD4 antibody that permits the binding of gp120 to the CD4 receptor but inhibits the infection of target cells by HIV-1 and the fusion inhibitor is selected from the group consisting of pentafuside and its functionally equivalent peptides.

6. The method of claim 1 further comprising exposing target cells to the attachment inhibitors and fusion inhibitors in combination with at least one other drug selected from the group consisting of integrase inhibitors, nucleoside reverse transcriptase inhibitors, non-nucleoside reverse transcriptase inhibitors, and HIV protease inhibitors.

7. The method of claim 1 wherein the attachment inhibitor is an anti-HIV-1 co-receptor antibody that binds to a chemokine co-receptor selected from the group consisting of CCR5 and CXCR4 and inhibits or prevents the attachment of the co-receptor to gp120 and the fusion inhibitor is selected from the group consisting of polypeptides that interact with gp41 to inhibit or prevent its harpoon-like attachment to target cells and polypeptides that interact with gp41 to inhibit or prevent its recoil-like action that brings HIV-1 into close contact with target cells.

8. The method of claim 7 wherein the fusion inhibitor is selected from the group consisting of anti-gp41 antibodies and polypeptides having from 30 to 50 amino acids and the ability to interact with gp41 to prevent its harpoon-like or recoil-like action.

9. The method of claim 7 wherein the fusion inhibitor is selected from the group consisting of T-1249, T-649, 5-Helix, pentafuside, and their functionally equivalent peptides.

10. The method of claim 7 wherein the fusion inhibitor is selected from the group consisting of pentafuside and its functionally equivalent peptides.

11. The method of claim 7 further comprising exposing target cells to the attachment inhibitors and fusion inhibitors in combination with at least one other drug selected from the group consisting of integrase inhibitors, nucleoside reverse transcriptase inhibitors, non-nucleoside reverse transcriptase inhibitors, and HIV protease inhibitors.

12. A method for preventing or treating acquired immunodeficiency syndrome (“AIDS”) comprising administering a disease preventing or treating amount of a synergistic combination of at least one attachment inhibitor and at least one fusion inhibitor to a patient at risk for contracting or suffering from AIDS.

13. The method of claim 12 wherein the attachment inhibitor is a polypeptide selected from the group consisting of polypeptides that bind to the CD4 receptor on target cells and inhibit or prevent HIV-1 from attaching to the target cell, polypeptides that bind to the CD4 receptor on target cells and permit HIV-1 to attach to the target cell but inhibit or prevent cellular fusion between HIV-1 and the target cell, polypeptides that bind to gp120 on HIV-1 and inhibit or prevent HIV-1 from attaching to the target cells, and polypeptides that bind to gp120 on HIV-1 and permit HIV-1 to attach to the target cell but inhibit or prevent cellular fusion between HIV-1 and the target cell and the fusion inhibitor is selected from the group consisting of polypeptides that interact with gp41 to inhibit or prevent its harpoon-like attachment to target cells and polypeptides that interact with gp41 to inhibit or prevent its recoil-like action that brings HIV-1 into close contact with target cells.

14. The method of claim 13 wherein the attachment inhibitor is selected from the group consisting of antibodies, antibody fragments, CD4 antagonists comprising a fragment of a CD4 ligand, and gp120 antagonists comprising a fragment of CD4 and the fusion inhibitor is selected from the group consisting of anti-gp41 antibodies and polypeptides having from 30 to 50 amino acids and the ability to interact with gp41 to prevent its harpoon-like or recoil-like action.

15. The method of claim 14 wherein the attachment inhibitor is selected from the group consisting of anti-CD4 antibodies and anti-gp120 antibodies and the fusion inhibitor is selected from the group consisting of T-1249, T-649, 5-Helix, pentafuside, and their functionally equivalent peptides.

16. The method of claim 13 wherein the attachment inhibitor is an anti-CD4 antibody that permits the binding of gp120 to the CD4 receptor but inhibits the infection of target cells by HIV-1 and the fusion inhibitor is selected from the group consisting of pentafuside and its functionally equivalent peptides.

17. The method of claim 12 further comprising exposing target cells to the attachment inhibitors and fusion inhibitors in combination with at least one other drug selected from the group consisting of integrase inhibitors, nucleoside reverse transcriptase inhibitors, non-nucleoside reverse transcriptase inhibitors, and HIV protease inhibitors.

18. The method of claim 12 wherein the attachment inhibitor is an anti-HIV-1 co-receptor antibody that binds to a chemokine co-receptor selected from the group consisting of CCR5 and CXCR4 and inhibits or prevents the attachment of the co-receptor to gp120 and the fusion inhibitor is selected from the group consisting of polypeptides that interact with gp41 to inhibit or prevent its harpoon-like attachment to target cells and polypeptides that interact with gp41 to inhibit or prevent its recoil-like action that brings HIV-1 into close contact with target cells.

19. The method of claim 18 wherein the fusion inhibitor is selected from the group consisting of anti-gp41 antibodies and polypeptides having from 30 to 50 amino acids and the ability to interact with gp41 to prevent its harpoon-like or recoil-like action.

20. The method of claim 18 wherein the fusion inhibitor is selected from the group consisting of T-1249, T-649, 5-Helix, pentafuside, and their functionally equivalent peptides.

21. The method of claim 18 wherein the fusion inhibitor is selected from the group consisting of pentafuside and its functionally equivalent peptides.

22. The method of claim 18 further comprising exposing target cells to the attachment inhibitors and fusion inhibitors in combination with at least one other drug selected from the group consisting of integrase inhibitors, nucleoside reverse transcriptase inhibitors, non-nucleoside reverse transcriptase inhibitors, and HIV protease inhibitors.

23. The method of claim 12 wherein the attachment inhibitor is administered to the patient in amounts of from about 1 to 50 milligrams per kilogram of body weight and the fusion inhibitor is administered to the patient in amounts of 0.1 to 10 milligrams per kilogram of body weight.

24. A composition for preventing infection of target cells by HIV-1 and for preventing or treating AIDS comprising at least one attachment inhibitor and at least one fusion inhibitor.

25. The composition of claim 24 wherein the attachment inhibitor is a polypeptide selected from the group consisting of polypeptides that bind to the CD4 receptor on target cells and inhibit or prevent HIV-1 from attaching to the target cell, polypeptides that bind to the CD4 receptor on target cells and permit HIV-1 to attach to the target cell but inhibit or prevent cellular fusion between HIV-1 and the target cell, polypeptides that bind to gp120 on HIV-1 and inhibit or prevent HIV-1 from attaching to the target cells, and polypeptides that bind to gp120 on HIV-1 and permit HIV-1 to attach to the target cell but inhibit or prevent cellular fusion between HIV-1 and the target cell and the fusion inhibitor is selected from the group consisting of polypeptides that interact with gp41 to inhibit or prevent its harpoon-like attachment to target cells and polypeptides that interact with gp41 to inhibit or prevent its recoil-like action that brings HIV-1 into close contact with target cells.

26. The composition of claim 25 wherein the attachment inhibitor is selected from the group consisting of antibodies, antibody fragments, CD4 antagonists comprising a fragment of a CD4 ligand, and gp120 antagonists comprising a fragment of CD4 and the fusion inhibitor is selected from the group consisting of anti-gp41 antibodies and polypeptides having from 30 to 50 amino acids and the ability to interact with gp41 to prevent its harpoon-like or recoil-like action.

27. The composition of claim 26 wherein the attachment inhibitor is selected from the group consisting of anti-CD4 antibodies and anti-gp120 antibodies and the fusion inhibitor is selected from the group consisting of T-1249, T-649, 5-Helix, pentafuside, and their functionally equivalent peptides.

28. The composition of claim 25 wherein the attachment inhibitor is an anti-CD4 antibody that permits the binding of gp120 to the CD4 receptor but inhibits the infection of target cells by HIV-1 and the fusion inhibitor is selected from the group consisting of pentafuside and its functionally equivalent peptides.

29. The composition of claim 24 further comprising at least one other drug selected from the group consisting of integrase inhibitors, nucleoside reverse transcriptase inhibitors, non-nucleoside reverse transcriptase inhibitors, and HIV protease inhibitors.

30. The composition of claim 24 wherein the attachment inhibitor is an anti-HIV-1 co-receptor antibody that binds to a chemokine co-receptor selected from the group consisting of CCR5 and CXCR4 and inhibits or prevents the attachment of the co-receptor to gp120 and the fusion inhibitor is selected from the group consisting of polypeptides that interact with gp41 to inhibit or prevent its harpoon-like attachment to target cells and polypeptides that interact with gp41 to inhibit or prevent its recoil-like action that brings HIV-1 into close contact with target cells.

31. The composition of claim 30 wherein the fusion inhibitor is selected from the group consisting of anti-gp41 antibodies and polypeptides having from 30 to 50 amino acids and the ability to interact with gp41 to prevent its harpoon-like or recoil-like action.

32. The composition of claim 30 wherein the fusion inhibitor is selected from the group consisting of T-1249, T-649, 5-Helix, pentafuside, and their functionally equivalent peptides.

33. The composition of claim 30 wherein the fusion inhibitor is selected from the group consisting of pentafuside and its functionally equivalent peptides.

34. The composition of claim 30 further comprising at least one other drug selected from the group consisting of integrase inhibitors, nucleoside reverse transcriptase inhibitors, non-nucleoside reverse transcriptase inhibitors, and HIV protease inhibitors.

35. An article of manufacture in the form of a kit comprising in separate containers in a single package a combination of two or more of an attachment inhibitor and a fusion inhibitor.

36. The article of manufacture of claim 35 further comprising another drug useful for inhibiting or preventing HIV-1 infection of target cells or for the prevention or treatment of AIDS.

37. The article of manufacture of claim 36 wherein the drug is selected from the group consisting of integrase, transcriptase, or protease inhibitors.

38. The article of manufacture of claim 35 wherein the attachment inhibitor is selected from the group consisting of anti-CD4 antibodies and anti-gp120 antibodies and the fusion inhibitor is selected from the group consisting of pentafuside and its functionally equivalent peptides.

39. A means for communicating information about or instructions for synergistically using attachment inhibitors and fusion inhibitors to prevent infection of target cells by HIV-1 and to prevent or treat AIDS comprising a document or visual display that contains the information or instructions.

40. The means of claim 39 selected from the group consisting of a web site displayed on a visual monitor, brochure, and or package insert.
